(19) United States
(12) Patent Application Publication
(10) Pub. No.: US 2012/0259234 A1
(43) Pub. Date: Oct. 11, 2012

(54) METHOD AND DEVICE FOR DETECTING INCIPIENT A-V NODE MALFUNCTION

(75) Inventor: Anders Lindgren, Taby (SE)

(73) Assignee: ST. JUDE MEDICAL AB, Jarfalla (SE)

(21) Appl. No.: 13/516,178

(22) PCT Filed: Dec. 22, 2009
(86) PCT No.: PCT/EP09/67787

§ 371 Date: Jun. 14, 2012

(51) Int. Cl. A61N 1/365; A61B 5/0472; A61B 5/0205; A61B 5/0452

(52) U.S. Cl. 600/484; 600/510; 600/509; 607/18

(57) ABSTRACT

The present invention generally relates to implantable stimulation devices, such as pacemakers, defibrillators, and cardioverters, and, in particular, to implantable medical devices using atrial based pacing such as an AAI pacing mode and methods for such implantable medical devices for detecting early stages of incipient A-V node malfunction as well as presence of A-V node malfunction. An AV conduction capacity is detected, wherein a sensed ventricular event following an intrinsic or paced atrial event during a predetermined period of time indicates good AV conduction capacity and wherein absence of a ventricular event within the predetermined period of time indicates poor AV conduction capacity. At least one A-V node function parameter indicating a function of the A-V node is determined, wherein the A-V node function parameter includes whether a status of the AV conduction capacity is good or poor. Incipient A-V node malfunction is detected where poor AV conduction capacity indicates incipient A-V node malfunction.

### TECHNICAL FIELD

The present invention generally relates to implantable stimulation devices, such as pacemakers, defibrillators, and cardioverters, and, in particular, to implantable medical devices using atrial based pacing such as an AAI pacing mode and methods for such implantable medical devices for detecting early stages of incipient A-V node malfunction as well as presence of A-V node malfunction.

### BACKGROUND OF THE INVENTION

Implantable cardiac stimulation devices such as pacemakers, defibrillators, and cardioverters are designed to monitor heart function and to rectify abnormal heart rhythms and/or contraction sequences/delays by delivering appropriately timed electrical stimulation signals. Using leads connected to a patient's heart, these devices typically stimulate the cardiac muscles by delivering electrical pulses in response to detected cardiac events which are indicative of the function of the heart. Properly administered therapeutical electrical pulses often successfully reestablish or maintain appropriate heart regular rhythm.

However, many traditional stimulation devices are unnecessarily designed to also pace in the ventricle and carries then additional redundant ventricular sensing/pacing hardware. Studies have shown that inappropriate ventricular pacing may have negative short-term and long-term hemodynamic effects and is thus not desirable when allowed to continue for an extended period of time. Several devices designed to reduce unnecessary pacing in the ventricle have been developed. Such devices are often designed to switch between an atrial based pacing mode (e.g. ADI or AAI) and a dual chamber pacing mode (e.g. DDD or DDI) to minimize the delivered ventricular pacing. In U.S. Pat. Appl. 2006/0247705, AV conduction block is monitored and if intrinsic R-waves are detected, the atrial based pacing mode is used and in absence of sensed ventricular events the ventricular pacing mode is used.

Commonly, these mode switching devices are not designed to anticipate or predict AV conduction blocks but instead to detect occurring AV conduction blocks. More specifically, the devices are not designed to anticipate or detect incipient malfunction of the A-V (atrioventricular) node function or early signs of imminent malfunction of the A-V node function that eventually may cause AV conduction blocks. A problem within the art is thus to predict whether an AV conduction block will arise or not or, in other words, to predict possible malfunction of the A-V node.

The A-V node has three main tasks. A first is to conduct depolarization, after a given period of time, from the atria to the ventricles. Since the ventricles are not depolarized immediately but a short time after the atria, the atria have sufficient time to discharge their blood contents into the ventricles. Conduction time is controlled by the autonomic nervous system. Increased sympathetic activity reduces conduction time, whereas increased parasympathetic activity has the opposite effect. A second task is to serve as a barrier to prevent the conduction of an excessive number of impulses per unit of time from the atria to the ventricles. Atrial fibrillation is therefore prevented from instigating conducted ventricular fibrillation. The A-V node's ability to protect against transmission of abnormally high atrial rate is related to its relatively long refractory time. Thirdly, the A-V node may serve as a back-up pacemaker if impulses from higher parts of the conduction system should be blocked.

A well accepted fact within the art is that atrial based pacing modes such as an AAI mode is preferable to ventricular single chamber pacing modes such as a WI mode. Consequently, the function of the A-V node and the stability of the function of the A-V node are essential in a decision whether an atrial based pacing mode, a dual chamber pacing mode or a combination of the pacing modes is the most suitable for a patient. The major concern by far with atrial based pacing modes such as AAI is that malfunction of the A-V node function cannot be anticipated or predicted with any accuracy or reliability, and, hence, it would be of great value if an incipient malfunction of the A-V node could be predicted in an accurate and reliable way.

### SUMMARY OF THE INVENTION

An object of the present invention is to provide methods and devices for detecting incipient malfunction of the A-V node in an accurate and reliable way.

This and other objects of the present invention are achieved by means of a method and an implantable medical device having the features defined in the independent claims. Different embodiments of the invention are characterized by the dependent claims.

According to an aspect of the present invention, there is provided a method for detecting incipient A-V node malfunction of a patient. The method comprises sensing far-field ventricular events and intrinsic or paced atrial events and determining whether a ventricular event following intrinsic or paced atrial events is sensed during a predetermined period of time. Further, an AV conduction capacity is detected based on whether ventricular events following intrinsic or paced atrial events are sensed, wherein a sensed ventricular event following an intrinsic or paced atrial event during a predetermined period of time indicates good AV conduction capacity and wherein absence of a ventricular event within the predetermined period of time indicates poor AV conduction capacity. At least one A-V node function parameter indicating a function of the A-V node is determined, wherein the A-V node function parameter includes whether a status of the AV conduction capacity is good or poor. Incipient A-V node malfunction based on the A-V node function parameter is detected. Poor AV conduction capacity indicates incipient A-V node malfunction.

According to another aspect of the present invention, there is provided an implantable medical device capable of detecting incipient A-V node malfunction of a patient. The device is connectable to at least one sensor for sensing far-field ventricular events and intrinsic or paced atrial events. Further, the device includes a cardiac event detection module adapted to determine whether a ventricular event following intrinsic or paced atrial events is sensed during a predetermined period of time, and to detect an AV conduction capacity based on whether ventricular events following intrinsic or paced atrial events are sensed, wherein a sensed ventricular event following an intrinsic or paced atrial event during a predetermined period of time indicates good AV conduction capacity and wherein absence of a ventricular event within the predetermined period of time indicates poor AV conduction capacity. An A-V node function detection module adapted to determine at least one A-V node function parameter indicating a function of the A-V node, the A-V node function parameter including whether a status of the AV conduction capacity is good or poor and to detect incipient A-V node malfunction based on the A-V node function parameter.

Hence, the present invention is based on the insights that A-V node malfunction can be detected or predicted on an early stage by monitoring an A-V conduction capacity and by creating an A-V node function parameter reflecting that A-V conduction capacity. Incipient A-V node malfunction is manifested by an abnormal A-V conduction in that a poor A-V conduction capacity indicates such incipient A-V node malfunction. In particular, the present invention is based on findings with regard to specific signs or evidence of debuting A-V node malfunction. A lowering Wenckebachpoint, i.e. that the atrial rate at which the A-V node start prolonging, is an early and reliable signal of debuting malfunction of the A-V node. Further, an increasing atrioventricular conduction time at a given atrial rate is also an early and reliable signal of debuting malfunction of the A-V node. Moreover, intermittently blocked atrial events are an early and reliable signal of debuting malfunction of the A-V node and an increased frequency and/or number of such intermittently blocked atrial events is an evident marker of incipient malfunction. By analyzing the atrial electrogram, ventricular depolarization's giving rise to far-field R-waves can be identified. Therefore, it is possible to, via the atrial electrogram, to detect whether an atrial depolarization was successfully conducted to the ventricles and if the conduction time was normal at a certain paced/sensed atrial rate, i.e. whether the A-V conductions capacity was good or poor. According to the present invention, the atrial electrogram is continuously monitored to determine whether a far-field R-wave follows each atrial event. Further, it has also been found that the morphology of the far-field ventricular signal (or IEGM signal) can be used to detect early signs of incipient A-V node malfunction. In particular, the inventor has found that a broadened QRS-complex is a sign of incipient A-V node malfunction. It is believed that this is caused by so called bundle branch block. By comparing present signal morphologies with reference morphologies for the specific patient, it is possible to identify or detect small changes in the QRS complex and, in particular, whether the QRS-complex shows any signs of a broadening.

In one embodiment of the present invention, the method for detecting incipient A-V node malfunction of a patient includes determining at least one A-V node function parameter indicating the function of the A-V node, wherein the A-V node function parameter is the atrial rate at which the AV conduction capacity becomes poor. That is, the A-V node function parameter is the Wenckebachpoint, which is the atrial rate at which the A-V node starts blocking. Incipient A-V node malfunction is detected by comparing the A-V node function parameter with a reference atrial rate at which the AV conduction capacity becomes poor, wherein an A-V node function parameter being below the reference indicates an incipient A-V node malfunction. The reference Wenckebachpoint may be determined, for example, based on patient specific measurement or from statistics over measurements from groups of patients. Preferably, the reference Wenckebachpoint is obtained from the patient during conditions where it is established that the AV conduction capacity is good. A lowering Wenckebachpoint is an early indicator of incipient A-V node malfunction and therefore a comparison with the reference Wenckebachpoint obtained at an established good AV conduction capacity provides a reliable and early indicator of incipient A-V node malfunction.

According to a further embodiment of the present invention, at least one A-V node function parameter indicating the function of the A-V node at predetermined intervals, e.g. at specific time points at regular intervals, is determined, wherein the A-V node function parameter includes an atrial rate at which the AV conduction capacity becomes poor. The A-V node function parameter is the Wenckebachpoint, which is the atrial rate at which the A-V node starts blocking. Incipient A-V node malfunction is detected by monitoring the A-V node function parameter over time, wherein a lowering A-V node function parameter over time indicates an incipient A-V node malfunction. Hence, the Wenckebachpoint is monitored over time in order to identify small changes indicating worsening conduction status, i.e. whether the Wenckebachpoint is gradually lowered. Thereby, it may be possible to identify incipient A-V node malfunction at a really early stage since the Wenckebachpoint may be gradually lowered at a level being above the reference Wenckebachpoint discussed above. In one embodiment of the present invention, the rate of change of the Wenckebachpoint is also studied to detect incipient A-V node malfunction. An increased rate of change of the Wenckebachpoint may be an early sign of an incipient A-V node malfunction. Consequently, the trend of the Wenckebachpoint over time is studied and changes in the trend, and, in particular, changes that indicate a lowering Wenckebach point are evaluated to detect whether these changes indicate incipient A-V node malfunction.

In an embodiment of the present invention, a PR or AR interval is determined based on sensed ventricular and intrinsic or paced atrial events. At least one A-V node function parameter indicating the function of the A-V node is determined, wherein the at least one A-V node function parameter includes the PR or AR interval as a function of atrial rate. Incipient A-V node malfunction is detected by comparing the A-V node function parameter with a reference PR or AR interval at a present atrial rate, wherein an A-V node function parameter exceeding the reference indicates incipient A-V node malfunction. An increasing atrioventricular conduction time at a given atrial rate is a reliable and early sign of incipient A-V node malfunction, which is utilized in this embodiment of the invention. A present PR or AR interval, at a given atrial rate, exceeding the reference interval is thus a reliable and early sign of incipient A-V node malfunction. The reference PR or AR interval as a function of atrial rate may be determined, for example, based on patient specific measurement or from statistics over measurements from groups of patients. Preferably, the reference PR or AR interval as a function of atrial rate is obtained from the patient during conditions where it is established that the AV conduction capacity is good. The reference PR or AR interval is in fact a number of reference intervals for different atrial rates. Thereby, a present A-V node function parameter at a specific atrial rate can be compared with the reference interval for that specific atrial rate. In one embodiment, incipient A-V node malfunction is detected if it is determined that the A-V node parameter exceeds the reference at one specific atrial rate. In other embodiments, several atrial rates are studied, for example, and A-V node function parameters at different atrial rates may be associated with different weights. It may for example be more interesting to study normal atrial rates, i.e. atrial rates at everyday activity of the patient, than atrial rates at rest. Incipient A-V node malfunction can be detected if a weighted A-V node function parameter over a number of different atrial rates (the weighted A-V node function parameter is the sum of the A-V node function parameters with a certain weight for the different atrial rates, or in other words, the weighted conduction time is the sum of a number of conduction times) exceeds the corresponding reference.

According to an embodiment of the present invention, PR or AR intervals are determined based on sensed ventricular and intrinsic or paced atrial events at predetermined intervals, e.g. at specific time points at regular intervals. At least one A-V node function parameter indicating the function of the A-V node for consecutive cardiac cycles is determined, wherein the at least one A-V node function parameter is the PR or AR interval as a function of atrial rate. That is, the atrioventricular conduction time as a function of the atrial rate is used as A-V node function parameter. One A-V node function parameter can be determined for each cardiac cycle, or the A-V node function parameter can be determined based on, for example, an average of a number of preceding intervals. A two-dimensional matrix of PR or AR intervals over time at different atrial rates can thus be created. Incipient A-V node malfunction is detected by monitoring the A-V node function parameter over time, at specific atrial rates, wherein an increasing A-V node function parameter over time indicates incipient A-V node malfunction. That is, an increasing atrioventricular conduction time at a specific atrial rate over time indicates incipient A-V node malfunction. The atrioventricular conduction time is monitored over time in order to identify small changes indicating worsening conduction status, i.e. whether the atrioventricular conduction time is gradually increased. Thereby, it may be possible to identify incipient A-V node malfunction at a really early stage since the atrioventricular conduction time may be gradually increased at a level above the reference discussed above. In one embodiment of the present invention, the rate of change of the atrioventricular conduction time is also studied to detect incipient A-V node malfunction. An increased rate of change of the atrioventricular conduction time may be an early sign of an incipient A-V node malfunction. Consequently, the trend of the atrioventricular conduction time over time is studied and changes in the trend, and, in particular, changes that indicate an increasing atrioventricular conduction time are evaluated to detect whether these changes indicate incipient A-V node malfunction.

Further, the gradual changes may only be observed for specific atrial rates.
In one embodiment, incipient A-V node malfunction is detected if it is determined that the A-V node parameter at one specific atrial rate increases over time. In other embodiments, several atrial rates are studied and, for example, A-V node function parameters at different atrial rates may be associated with different weights. It may for example be more interesting to study normal atrial rates, i.e. atrial rates at everyday activity of the patient, than atrial rates at rest. Incipient A-V node malfunction can be detected if a weighted A-V node function parameter over a number of different atrial rates (the weighted A-V node function parameter is the sum of the A-V node function parameters with a certain weight for the different atrial rates, or in other words, the weighted conduction time is the sum of a number of conduction times) increases over time.

In a further embodiment of the present invention, an A-V node function parameter including whether a status of the AV conduction capacity is good or poor is determined. Incipient A-V node malfunction is detected when the A-V node function parameter indicates temporarily poor A-V conduction conditions. That is, intermittently blocked atrial events are used as an indicator of incipient A-V node malfunction. The frequency of the blocked atrial events may also be an early indicator of such incipient malfunction, and, in particular, an increased frequency over time or over a period of time is an indicator of incipient A-V node malfunction. Further, the number of blocked atrial events may also be an early indicator of such incipient malfunction, and, in particular, an increased number of blocked atrial events during a predetermined period of time are an indicator of incipient A-V node malfunction.

According to an embodiment of the present invention, the morphology of the sensed ventricular events is determined. For example, the morphology for each sensed ventricular event can be determined, i.e. for each cardiac cycle where a ventricular event is sensed. The morphology is analysed to determine A-V node function indicating characteristics and at least one A-V node function parameter indicating the function of the A-V node for consecutive cardiac cycles is determined, wherein the at least one A-V node function parameter includes the A-V node function indicating characteristics. Incipient A-V node malfunction is detected by monitoring the A-V node function parameter over time, wherein an A-V node function parameter deviating from a reference A-V node function parameter over time indicates incipient A-V node malfunction. For example, the A-V node indicating characteristics may be a curve shape of the QRS-complex or a width of the QRS-complex at specific amplitude. Thus, by studying changes in the morphology of the far-field signal it is possible to identify early signs of incipient A-V node malfunction. The morphology can be monitored over time and/or can be compared with reference morphology for the patient. Preferably, the reference morphology is created using far-field signals measured during conditions where it is established that the AV conduction capacity is good. If the morphology is studied over time, small gradual changes can be captured, which may indicate incipient A-V node malfunction. A rate of change may also be monitored and an increased rate of change may be an early indicator of A-V node malfunction.

In one embodiment of the present invention, the morphology is analysed to determine A-V node function indicating characteristics, wherein the A-V node function indicating characteristics are a width of a QRS-complex at a specific amplitude. At least one A-V node function parameter indicating the function of the A-V node is determined, wherein the at least one A-V node function parameter includes the width of the QRS-complex. Incipient A-V node malfunction is detected by monitoring the A-V node function parameter over time, wherein an increasing A-V node function over time indicates incipient A-V node malfunction or wherein an A-V node function exceeding a reference A-V node parameter. Hence, this embodiment is based on the insight that an increased width of the QRS-complex indicates incipient A-V node malfunction. The increased width of the QRS-complex is caused by bundle branch block and this is an early and reliable sign of an incipient A-V node malfunction. This phenomenon can be studied over time and the earlier A-V node function parameters may serve as references in that a deviation in the width i.e. increased width, over time can indicate an incipient A-V node malfunction. If the morphology and the width of the QRS-complex is studied over time, small gradual changes can be captured, which may indicate incipient A-V node malfunction. A rate of change of the increased width of the QRS-complex may also be monitored and an increased rate of change of the increase may be an early indicator of A-V node malfunction.

According to an embodiment of the present invention, at least one physiological or hemodynamical parameter of the patient is sensed. This at least one physiological or hemodynamical parameter may be, for example, a heart rate of the patient, an activity level of the patient, or a breathing rate of the patient. If the physiological or hemodynamical parameter reaches a predetermined level, the atrial rate (the paced rate or the sensed rate) a predetermined amount is increased a predetermined amount by artificial stimulation via the pacemaker. Thus, the atrial rate is increased in order to test the A-V conduction capacity and to identify an atrial rate at which the A-V conduction capacity becomes poor. Thereby, it is possible to identify incipient A-V node malfunction at an early stage because the signs of such incipient malfunction at early stages may not be evident during normal conditions, for example, during normal activity of the patient but at certain conditions, which hence may be provoked by this procedure. For example, the A-V function parameter based on the Wenckebachpoint, and/or the atrioventricular conduction time, and/or morphology changes, and/or intermittently blocked atrial events (e.g. frequency and/or number of blocks) can be determined and used in the detection of incipient A-V node malfunction.

In one embodiment of the present invention, the activity level of the patient is sensed. For example, an accelerometer can be used to sense the activity of the patient. The heart rate or the breathing rate may also/additionally be used to determine the activity level. If the activity level exceeds a predetermined level, the atrial rate (the paced rate or the sensed rate) a predetermined amount is increased a predetermined amount by artificial stimulation via the pacemaker. Thus, the atrial rate is increased in order to test the A-V conduction capacity during a higher degree of activity of the patient to identify an atrial rate at which the A-V conduction capacity becomes poor. Thereby, it is possible to identify incipient A-V node malfunction at an early stage because the signs of such incipient malfunction at early stages may not be evident during normal activity of the patient but at increased activity such as during exercise. For example, the A-V function parameter based on the Wenckebachpoint, and/or the atrioventricular conduction time, and/or morphology changes, and/or intermittently blocked atrial events (e.g. frequency and/or number of blocks) can be determined and used in the detection of incipient A-V node malfunction.

In embodiment of the present invention, the patient and/or an external device, e.g. a home monitoring device or a monitoring device at a care provider, is notified if an incipient malfunction of the A-V node is detected by means of an alert signal. The alert signal may be sent wirelessly from the implantable medical device to the external device. Preferably, the patient is notified by issuing a perceptible alert signal, for example, the patient can be notified by means of a vibrating device connected to or arranged within the implantable medical device or by means of a sound emitting device emitting a tone.

As the skilled person realizes, steps of the methods according to the present invention, as well as preferred embodiments thereof, are suitable to realize as computer program or as a computer readable medium.

Further objects and advantages of the present invention will be discussed below by means of exemplifying embodiments.

### BRIEF DESCRIPTION OF THE DRAWINGS

Exemplifying embodiments of the invention will be described below with reference to the accompanying drawings, in which:

FIG. 1 is a simplified, partly cutaway view, illustrating an implantable medical device according to the present invention with a set of leads implanted into the heart of a patient;

FIG. 2 is a functional block diagram form of the implantable medical device shown in FIG. 1.

FIG. 3 schematically illustrates the principles of an embodiment of the method according to the present invention.

FIG. 4-8 schematically illustrating atrial electrograms showing normal A-V node function and indications of A-V node malfunction.

### DESCRIPTION OF EXEMPLIFYING EMBODIMENTS

The following is a description of exemplifying embodiments in accordance with the present invention. The present invention is preferably implemented in a pacemaker operating in an atrial based pacing mode such as an AAI mode. This description is however not to be taken in limiting sense, but is made merely for the purposes of describing the general principles of the invention. It is to be understood that other embodiments may be utilized and structural and logical changes may be made without departing from the scope of the present invention.

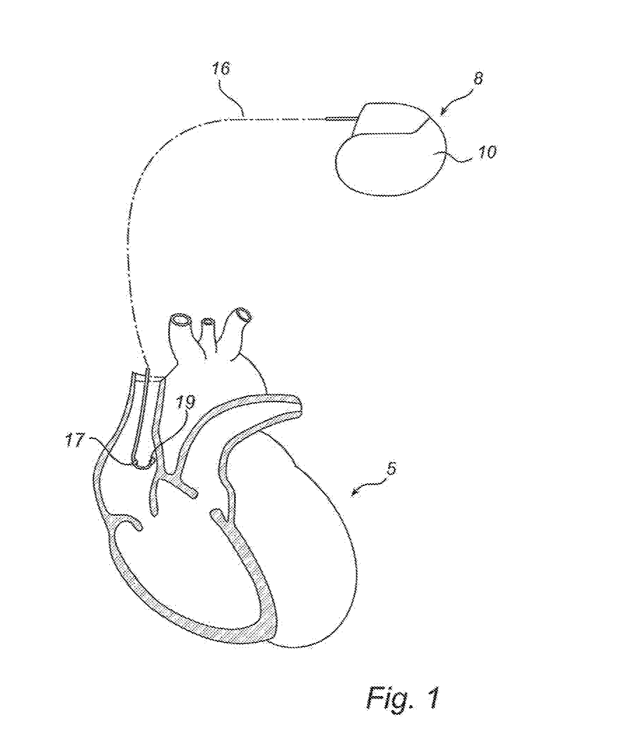

Turning now to FIG. 1, which is a simplified schematic view of one embodiment of an implantable medical device (“IMD”) 8 according to the present invention. IMD 8 has a hermetically sealed and biologically inert case 10. In this embodiment, IMD 8 is a pacemaker which is connectable to pacing and sensing lead 16, in this illustrated case one lead. However, as the skilled person understands, the pacemaker may also be connected to one or several, e.g. three or more, pacing and sensing leads. IMD 8 is in electrical communication with a patient's heart 5 via a right atrium (RA) lead 16 implanted in the atrial appendage having a RA tip electrode 19 and a RA ring electrode 17 is arranged to provide electrical communication between the right atrium (RA) and the IMD 8. With this configuration atrial based pacing can be performed. Although one medical lead is shown in FIG. 1, however, it should also be understood that additional stimulation leads (with one or more pacing, sensing, and/or shocking electrodes) may be used.

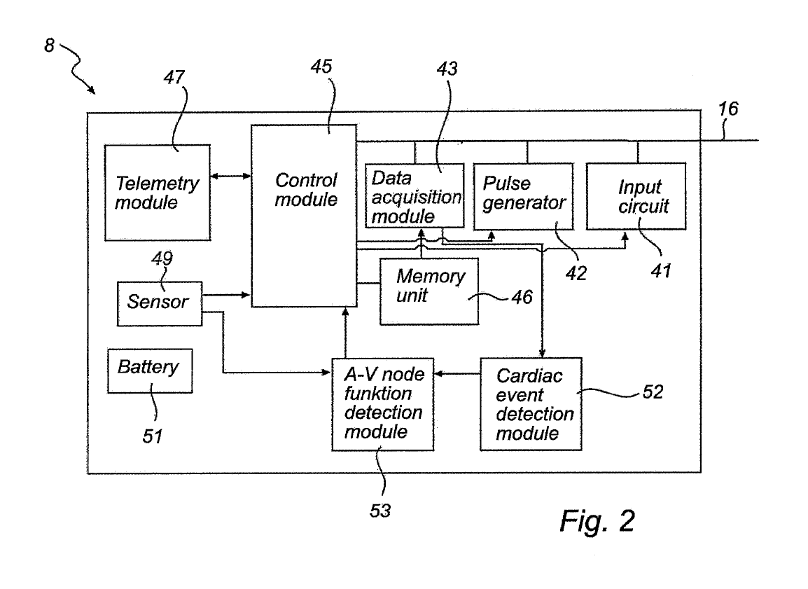

FIG. 2 is a block diagram illustrating the constituent components of an IMD 8 in accordance with the general principles of the present invention.

According to this embodiment, the IMD 8 is a pacemaker having a microprocessor based architecture. The lead 16 is connectable to the IMD 8 and comprises, as have been illustrated in FIG. 1, one or more electrodes, such a coils, tip electrodes or ring electrodes. The housing 10 (see FIG. 1) of the IMD 8, shown schematically in FIG. 2, is often referred to as the “can”, “case”, or “case electrode” and may be programmed to act as the return electrode in, for example, “unipolar” modes. The housing 10 further includes a connector (not shown) having a plurality of terminals (not shown) for connection to the medial lead 16 and the included electrodes 17 and 19. Thus, the lead 16 is connectable to the IMD 8 and comprises, as have been illustrated in FIG. 1, one or more electrodes, such a coils, tip electrodes or ring electrodes. These electrodes are arranged to, inter alia, transmit pacing pulses for causing depolarization of cardiac tissue adjacent to the electrode (-s) generated by a pace pulse generator 42 under influence of a control module or microcontroller 45. The rate of the heart 5 is controlled by software-implemented algorithms stored within a microcomputer circuit of the control module 45. As well known in the art, the microcomputer (also referred to as a microprocessor) of the control module is designed specifically for controlling the delivery of stimulation therapy and may further comprise random access memory (RAM) and read-only memory (ROM), logic and timing circuitry, state machine circuitry, and I/O circuitry. Typically, the control module 45 includes the ability to process or monitor input signals (data) as controlled by a program code stored in a designated block of memory. The details of design and operation of the control module 45 are not critical to the invention. Rather, any suitable control module 45 may be used that carries out the functions described herein. The use of microprocessor-based control circuits for performing timing and data analysis functions are well known in the art.

The pulse generator 42 includes an atrial pulse generator (not shown) adapted to generate pacing stimulation pulses for delivery by the right atrial lead 16 via an electrode configuration switch (not shown). The pulse generator 42 is controlled by the control module 45 via appropriate control signals to trigger or inhibit the stimulation pulses.

The control module 45 further includes timing control circuitry (not separately shown) used to control the timing of such stimulation pulses (e.g. pacing rate, atrial interconduction (A-A) delay etc.) as well as to keep track of the timing of refractory periods, blanking intervals, noise reduction windows, evoked response windows, alter intervals, marker channel timing, etc., which is well known in the art.

An input circuit 41 selectively coupled to the medical lead 16 includes atrial sensing circuits (not shown) for detecting the presence of cardiac activity in the right atrium and via far field signals in the other chambers of the heart. The sensing circuits may include sense amplifiers with programmable gain and/or automatic gain control, band-pass filtering, and a threshold detection circuit, as known in the art, to selectively sense cardiac signals. The outputs of the input circuit 41 are connected to the control module 45 which, in turn, is able to trigger or inhibit the pulse generator 42 in a demand fashion in response to the absence or presence of cardiac activity in the appropriate chamber of the heart. Further, the IMD 8 may use the sensing circuits of the input circuit 41 to sense cardiac signals to determine whether a rhythm is physiologic or pathologic in arrhythmia detection purposes.

A data acquisition module 43 including analog-to-digital converters is adapted to acquire analog intracardiac electrogram signals and convert the acquired analog signals to digital signals and store the signals for later processing and/or telemetric transmission to external devices in a memory unit 46. The data acquisition module 43 is coupled to the medical lead 16 to sample cardiac signal across for example of electrodes 17 and 19.

The control module 45 is also connected to the memory unit 46 via a suitable data/address bus (not shown), wherein operating parameters used by the control module 45 can be stored and modified as required, in order to customize the operation of the IMD 8 to suit the needs of a particular patient. Such operating parameters define, for example, pacing pulse amplitude or magnitude, pulse duration, electrode polarity, rate, sensitivity, automatic features, and arrhythmia detection criteria. Other parameters may include base rate, rest rate, and circadian base rate.

The operating parameters of the IMD 8 may be non-invasively programmed into the memory unit 46 through a communication module or telemetry module 47 comprising a receiver (not shown) and a transmitter (not shown) in telemetric communication with an external device such as a programmer, or the external monitoring system. The telemetry module 46 allows IEGMs and other physiological signal and/or hemodynamic signals as well as, for example, status information related to the operation of the IMD 8 to be sent to the external programmer and/or the external monitoring system through an established communication links. To facilitate communication with the external monitoring system and/or the external programmer MICS band components (not shown) and ISM band components (not shown) are provided within the telemetry module 47.

The IMD 8 may further include an activity sensor or other physiologic sensors 49, which may be used to adjust pacing stimulation rate according to the exercise state of the patient. However, the sensor 49 may further be used to detect changes in cardiac output or changes in physiological condition of the heart. While shown as being included within the IMD 8, it is to be understood that the sensor 49 may also be external to the IMD 8, yet still be implanted within or carried by the patient. A common type of activity sensor is an accelerometer or a piezoelectric crystal mounted within the housing. Other types of physiological sensors are also known, for example, sensors that sense the oxygen content of the blood, respiration rate and/or minute ventilation, pH of blood etc.

The IMD 8 additionally includes a battery 51, which provide operating power to all the circuits shown in FIG. 2 (in order not to burden the illustration the connections to the other circuits of FIG. 2 are not shown). The battery 51 may vary depending on the capabilities of the IMD 8. If the system provides low voltage therapy, a lithium iodine or lithium copper fluoride cell may be utilized.

Furthermore, the IMD 8 includes a cardiac event detection unit 52 connected to the data acquisition module 43 adapted to determine whether a ventricular event follows an intrinsic or paced atrial event or, in other words, whether a far-field ventricular event was sensed following the intrinsic or paced event. Further, the cardiac event detection unit 52 is adapted to detect an AV conduction capacity based on whether ventricular events following intrinsic or paced atrial events could be sensed. A sensed far-field ventricular event following an intrinsic or paced atrial event during a predetermined period of time is an indication of good AV conduction capacity, e.g. within a period of time of 200 ms. The absence of a far-field ventricular event within the predetermined period of time indicates poor AV conduction capacity.

The IMD 8 also includes an AV node function detection module 53 adapted to determine at least one A-V node function parameter. This parameter indicates a function of the A-V node and based on this parameter it is detected whether there are any signs of incipient A-V node malfunction being present.

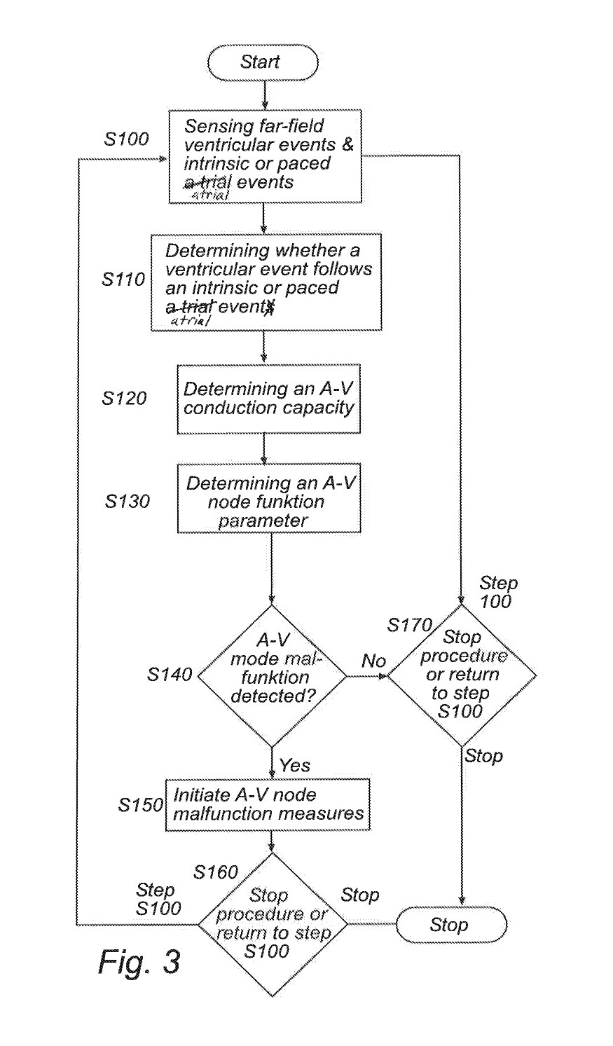

With reference now to FIG. 3, the overall principles of a method according to the present invention will be discussed.

The algorithm for detecting incipient malfunction of the A-V node may be used for continuous monitoring of the A-V node function or detection sessions can be initiated at regular intervals or upon receipt of an initiation signal, for example, from an external device. First, at step S100, far-field ventricular events and intrinsic or paced atrial events are sensed. At step S110, it is determined whether a ventricular event following intrinsic or paced atrial events is sensed during a predetermined period of time. Thereafter, at step S120, an AV conduction capacity is determined based on whether ventricular events following intrinsic or paced atrial events could be sensed.

A sensed ventricular event following an intrinsic or paced atrial event during a predetermined period of time indicates good AV conduction capacity. For example, a sensed ventricular event in each cardiac cycle may indicate good AV conduction capacity, and thus, a transient loss of AV conduction for at least one cardiac cycle indicates poor AV conduction capacity (or, in other words, an absence of a ventricular event within the predetermined period of time indicates poor AV conduction capacity). In FIG. 4, atrial electrogram showing A-V node block is illustrated. Subsequently, at step S130, it is at least one A-V node function parameter indicating a function of the A-V node is determined. In one embodiment of the present invention, the A-V node function parameter includes the AV conduction capacity. Below, a number of other embodiments will be described. At step S140, a detection step is performed in which the A-V node function parameter is used to detect incipient A-V node malfunction. That is, it is checked whether the A-V node function parameter indicates A-V node malfunction or not. This can be made, for example, by comparing the A-V node parameter with a reference parameter based on measurements of the patient, or based on statistical data from several patients, or by comparing the A-V node parameter with A-V function parameters determined earlier, i.e. comparisons over time. The measurements used for determining the A-V node function parameters can be made, for example, at a predetermined time point at regular intervals, e.g. a specific time point during each day, or at detection of specific conditions.

According to embodiments of the present invention, if it is detected that the A-V node function parameter indicates A-V node malfunction, the algorithm proceeds to step S150 where A-V node malfunction measures are taken. However, it should be noted that this step is optional and the procedure may, in other embodiments, proceed directly to step S160 or S170. In the optional step S150, a number of different measures may be taken. For example, the atrial electrogram documenting the elapsed event (i.e. the malfunction episode) can be saved and sent, via the telemetry module 47, to an external device. Further, the patient and/or physician may be notified of the event. The patient may be notified by means of an emitted tone or by vibration and the physician may be notified via an external device (e.g. via Merlin@home, which is a product manufactured by St. Jude Medical Inc.). Thereafter, at step S160, the algorithm may be stopped or may return to step S100, where the procedure re-starts or is resumed. If, at step S140, the A-V node function parameter indicates that the A-V node has a good function, the procedure may return to step S100 or may be stopped.

Hereinafter, number of different embodiments of the present invention will be discussed and, in particular, a number of different approaches for detecting such incipient A-V node malfunction.

According to an embodiment of the present invention, at least one A-V node function parameter including an atrial rate at or above which the AV conduction capacity becomes poor is determined. That is, in this embodiment, the A-V function parameter is the atrial rate at which the A-V node starts blocking atrial events, i.e. the so called Wenckebachpoint. A lowering Wenckebachpoint is evidence or sign of a debuting A-V node malfunction. Therefore, incipient A-V node malfunction can be detected by comparing the A-V node function parameter with a reference atrial rate at (above) which the AV conduction capacity becomes poor, wherein an A-V node function parameter being below the reference indicates an incipient A-V node malfunction. As discussed above, the reference parameter may be determined on basis of measurements performed in the patient or on basis of statistical data based on measurements from several patients. In FIG. 8, Wenckebach is illustrated. The distance or period of time between a P-wave and the subsequent far-field R-wave is gradually increased from x, to x+δ₁, to x+δ₂, etc. (δ₂>δ₁) until an atrial event is blocked and the sequence is restarted.

In another embodiment of the present invention, the A-V node function parameter is the Wenckebach point, which is the atrial rate at which the A-V node starts blocking. Incipient A-V node malfunction is detected by monitoring the A-V node function parameter over time. The measurements used for determining the Wenckebachpoint can be made, for example, at a predetermined time point at regular intervals, e.g. a specific time point during each day, or at detection of specific conditions. A lowering A-V node function parameter over time indicates an incipient A-V node malfunction, i.e. parameters determined based on earlier measurement function as the reference. Hence, the Wenckebachpoint is monitored over time in order to identify small changes indicating worsening conduction status, i.e. whether the Wenckebachpoint is gradually lowered. It is also possible to study, alternatively or as a complement, the rate of change of the Wenckebachpoint to detect incipient A-V node malfunction. An increased rate of change of the Wenckebachpoint may be an early sign of an incipient A-V node malfunction.

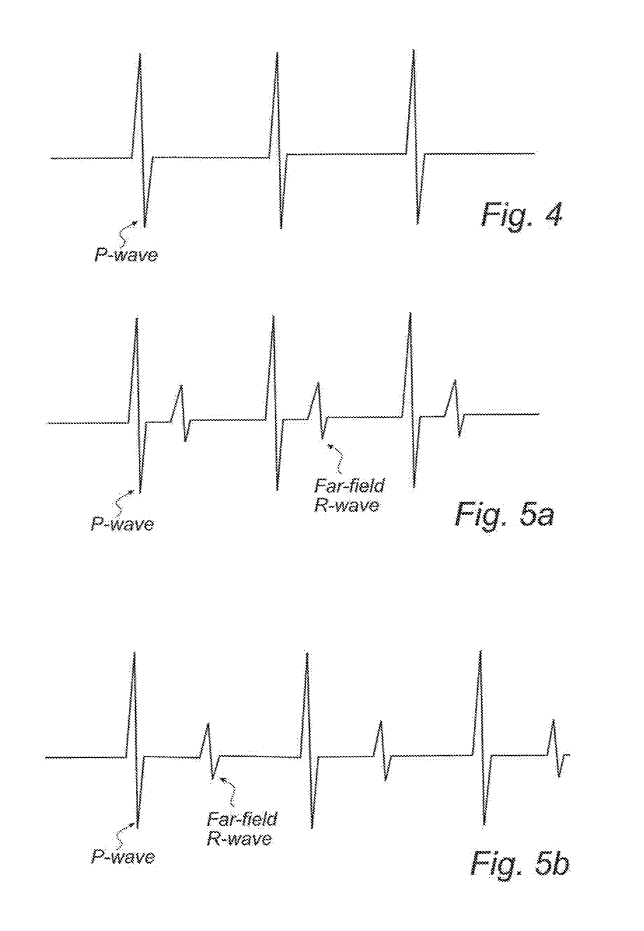

According to a further embodiment of the present invention, a PR or AR interval is determined based on sensed ventricular and intrinsic or paced atrial events. At least one A-V node function parameter indicating the function of the A-V node is determined, wherein the at least one A-V node function parameter includes the PR or AR interval as a function of atrial rate. Incipient A-V node malfunction is detected by comparing the A-V node function parameter with a reference PR or AR interval at a present atrial rate, wherein an A-V node function parameter exceeding the reference indicates incipient A-V node malfunction. An increasing atrioventricular conduction time at a given atrial rate is a reliable and early sign of incipient A-V node malfunction. In FIGS. 5a and 5b, schematic atrial electrograms illustrating this are shown. FIG. 5a illustrates normal A-V node function and FIG. 5b illustrates atrial electrograms showing a prolonged atrioventricular conduction time for a given atrial rate. A present PR or AR interval, at a given atrial rate, exceeding the reference interval is thus a reliable and early sign of incipient A-V node malfunction. The reference PR or AR interval as a function of atrial rate may be determined, for example, based on patient specific measurement or from statistics over measurements from groups of patients. Preferably, the reference PR or AR interval as a function of atrial rate is obtained from the patient during conditions where it is established that the AV conduction capacity is good. The reference PR or AR interval is in fact a number of reference intervals for different atrial rates. Thereby, a present A-V node function parameter at a specific atrial rate can be compared with the reference interval for that specific atrial rate. In one embodiment, incipient A-V node malfunction is detected if it is determined that the A-V node parameter exceeds the reference at one specific atrial rate. In other embodiments, several atrial rates are studied, for example, and A-V node function parameters at different atrial rates may be associated with different weights. It may for example be more interesting to study normal atrial rates, i.e. atrial rates at everyday activity of the patient, than atrial rates at rest. Incipient A-V node malfunction can be detected if a weighted A-V node function parameter over a number of different atrial rates (the weighted A-V node function parameter is the sum of the A-V node function parameters with a certain weight for the different atrial rates, or in other words, the weighted conduction time is the sum of a number of conduction times) exceeds the corresponding reference.

According to another embodiment of the present invention, PR or AR intervals are determined based on sensed ventricular and intrinsic or paced atrial events at predetermined intervals, e.g. at specific time points at regular intervals. At least one A-V node function parameter indicating the function of the A-V node for consecutive cardiac cycles is determined, wherein the at least one A-V node function parameter is the PR or AR interval as a function of atrial rate. That is, the atrioventricular conduction time as a function of the atrial rate is used as A-V node function parameter. One A-V node function parameter can be determined for each cardiac cycle, or the A-V node function parameter can be determined based on, for example, an average of a number of preceding intervals. A two-dimensional matrix of PR or AR intervals over time at different atrial rates can thus be created. Incipient A-V node malfunction is detected by monitoring the A-V node function parameter over time, at specific atrial rates, wherein an increasing A-V node function parameter over time indicates incipient A-V node malfunction. That is, an increasing atrioventricular conduction time at a specific atrial rate over time indicates incipient A-V node malfunction. The atrioventricular conduction time is monitored over time in order to identify small changes indicating worsening conduction status, i.e. whether the atrioventricular conduction time is gradually increased. The measurements used for determining the A-V node function parameters can be made, for example, at a predetermined time point at regular intervals, e.g. a specific time point during each day, or at detection of specific conditions. In one embodiment of the present invention, the rate of change of the atrioventricular conduction time is also studied to detect incipient A-V node malfunction. An increased rate of change of the atrioventricular conduction time may be an early sign of an incipient A-V node malfunction. Such gradual changes may only be observed for specific atrial rates and incipient A-V node malfunction may thus be detected if it is determined that the A-V node parameter at one specific atrial rate increases over time.

In a further embodiment of the present invention, an A-V node function parameter including whether a status of the AV conduction capacity is good or poor is determined. Incipient A-V node malfunction is detected when the A-V node function parameter indicates temporarily poor A-V conduction conditions. That is, intermittently blocked atrial events are used as an indicator of incipient A-V node malfunction. In FIG. 4, atrial electrogram showing A-V node block is illustrated. The frequency of the blocked atrial events may also be an early indicator of such incipient malfunction and an increased frequency over time or over a period of time may be an indicator of incipient A-V node malfunction. Further, the number of blocked atrial events may also be an early indicator of such incipient malfunction, and, in particular, an increased number of blocked atrial events during a predetermined period of time are an indicator of incipient A-V node malfunction.

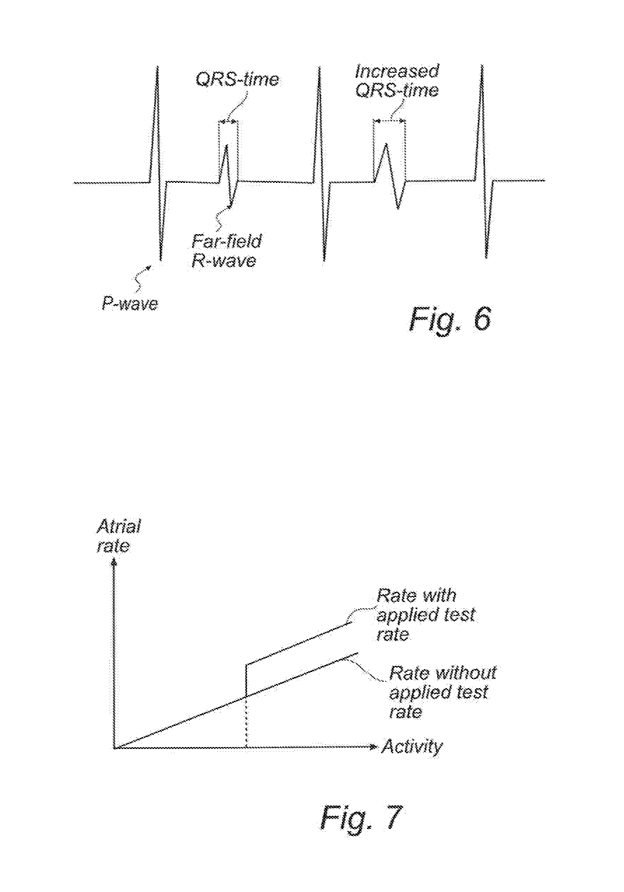

According to an embodiment of the present invention, the morphology of the sensed ventricular events is determined. For example, the morphology for each sensed ventricular event can be determined, i.e. for each cardiac cycle where a ventricular event is sensed. The morphology is analysed to determine A-V node function indicating characteristics and at least one A-V node function parameter indicating the function of the A-V node for consecutive cardiac cycles is determined, wherein the at least one A-V node function parameter includes the A-V node function indicating characteristics. Incipient A-V node malfunction is detected by monitoring the A-V node function parameter over time, wherein an A-V node function parameter deviating from a reference A-V node function parameter over time indicates incipient A-V node malfunction. For example, the A-V node indicating characteristics may be a curve shape of the QRS-complex or a width of the QRS-complex at specific amplitude. In FIG. 6, a broadened QRS-complex (i.e. increased QRS-time) is illustrated. An increased width of the QRS-complex indicates incipient A-V node malfunction. The increased width of the QRS-complex is caused by bundle branch block and this is an early sign of an incipient A-V node malfunction. Thus, by studying changes in the morphology of the far-field signal it is possible to identify early signs of incipient A-V node malfunction. The morphology can be monitored over time and/or can be compared with reference morphology for the patient. For example, measurements for obtaining the morphology may be performed at regular intervals, for example, each day at specific time points. Preferably, the reference morphology is created using far-field signals measured during conditions where it is established that the AV conduction capacity is good. If the morphology is studied over time, small gradual changes can be captured, which may indicate incipient A-V node malfunction. A rate of change may also be monitored and an increased rate of change may be an early indicator of A-V node malfunction.

According to yet another embodiment of the present invention, at least one physiological or hemodynamical parameter of the patient is sensed. This at least one physiological or hemodynamical parameter may be, for example, a heart rate of the patient, an activity level of the patient, or a breathing rate of the patient. If the physiological or hemodynamical parameter reaches a predetermined level, the atrial rate (the paced rate or the sensed rate) is increased a predetermined amount by artificial stimulation via the pacemaker. Thus, the atrial rate is increased in order to test the A-V conduction capacity and to identify an atrial rate at which the A-V conduction capacity becomes poor. Thereby, it is possible to identify incipient A-V node malfunction at an early stage because the signs of such incipient malfunction at early stages may not be evident during normal conditions, for example, during normal activity of the patient but at certain conditions, which hence may be provoked by this procedure. For example, the A-V function parameter based on the Wenckebachpoint, and/or the atrioventricular conduction time, and/or morphology changes, and/or intermittently blocked atrial events (e.g. frequency and/or number of blocks) can be determined and used in the detection of incipient A-V node malfunction.

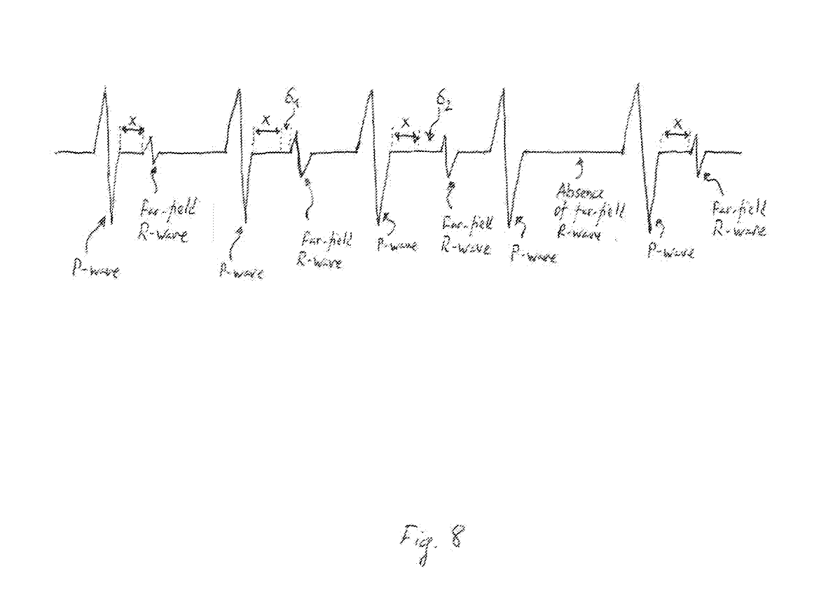

In one specific embodiment, the activity level of the patient is sensed. For example, the accelerometer 49 can be used to sense the activity of the patient. The heart rate or the breathing rate may also/additionally be used to determine the activity level. If the activity level exceeds a predetermined level, the atrial rate (the paced rate or the sensed rate) is increased a predetermined amount by artificial stimulation via the pacemaker. Thus, at a certain activity level, the atrial rate is increased a predetermined amount in order to test the A-V conduction capacity during a higher degree of activity of the patient to identify an atrial rate at which the A-V conduction capacity becomes poor. This is illustrated in FIG. 7.

This enables detection of incipient A-V node malfunction using anyone of the embodiment described above at an early stage because the signs of such incipient malfunction at early stages may not be evident during normal activity of the patient but at increased activity such as during exercise. For example, the A-V function parameter based on the Wenckebachpoint, and/or the atrioventricular conduction time, and/or morphology changes, and/or intermittently blocked atrial events (e.g. frequency and/or number of blocks) can be determined and used in the detection of incipient A-V node malfunction.

Although modifications and changes may be suggested by those skilled din the art, it is intended that all changes and modifications as reasonably and properly come within the scope of the contribution to the art made by this invention is embodied in the description and in the claims.

1. A method for detecting incipient A-V node malfunction of a patient comprising:
sensing far-field ventricular events and intrinsic or paced atrial events;
determining whether a far field ventricular event following intrinsic or paced atrial events is sensed during a predetermined period of time;
detecting AV conduction capacity based on whether far field ventricular events following intrinsic or paced atrial events are sensed, wherein a sensed far field ventricular event following an intrinsic or paced atrial event during a predetermined period of time indicates good AV conduction capacity and wherein absence of a far field ventricular event within the predetermined period of time indicates poor AV conduction capacity;
determining at least one A-V node function parameter indicating a function of the A-V node, said A-V node function parameter including whether a status of said AV conduction capacity is good or poor; and
detecting incipient A-V node malfunction based on said A-V node function parameter.

2. The method for detecting incipient A-V node malfunction of a patient according to claim 1, further comprising:
determining at least one A-V node function parameter indicating a function of the A-V node, said A-V node function parameter including a present paced or sensed atrial rate; and
detecting incipient A-V node malfunction based on said A-V node function parameter.

3. The method for detecting incipient A-V node malfunction of a patient according to claim 1, further comprising:
determining at least one A-V node function parameter indicating the function of the A-V node, wherein said A-V node function parameter includes an atrial rate above which the AV conduction capacity becomes poor; and
detecting incipient A-V node malfunction by comparing said A-V node function parameter with a reference atrial rate above which the AV conduction capacity becomes poor, wherein an A-V node function parameter being below said reference indicates an incipient A-V node malfunction.

4. The method for detecting incipient A-V node malfunction of a patient according to claim 3, further comprising:
determining at least one A-V node function parameter indicating the function of the A-V node at predetermined intervals, wherein said A-V node function parameter includes an atrial rate above which the AV conduction capacity becomes poor; and
detecting incipient A-V node malfunction by monitoring said A-V node function parameter over time, wherein a lowering A-V node function parameter over time indicates an incipient A-V node malfunction.

5. The method for detecting incipient A-V node malfunction of a patient according to claim 1, further comprising:
determining a PR or AR interval based on sensed ventricular and intrinsic or paced atrial events;
determining said at least one A-V node function parameter indicating the function of the A-V node, wherein said at least one A-V node function parameter includes said PR or AR interval as a function of atrial rate; and
detecting incipient A-V node malfunction by comparing said A-V node function parameter with a reference PR or AR interval at a present atrial rate, wherein an A-V node function parameter exceeding said reference indicates incipient A-V node malfunction.

6. The method for detecting incipient A-V node malfunction of a patient according to claim 5, further comprising:
determining PR or AR intervals based on sensed ventricular
and intrinsic or paced atrial events at predetermined intervals;
determining said at least one A-V node function parameter
indicating the function of the A-V node for consecutive cardiac cycles, wherein said at least one A-V node function parameter includes said PR or AR interval as a function of atrial rate; and
detecting incipient A-V node malfunction by monitoring said A-V node function parameter over time, at specific atrial rates, wherein an increasing A-V node function parameter over time indicates incipient A-V node malfunction.

7. The method for detecting incipient A-V node malfunction of a patient according to claim 1, further comprising:
determining a morphology of said sensed ventricular events;
analysing said morphology to determine A-V node function indicating characteristics;
determining at least one A-V node function parameter indicating
the function of the A-V node for consecutive cardiac cycles, wherein said at least one A-V node function parameter includes said A-V node function indicating characteristics; and
detecting incipient A-V node malfunction by monitoring said A-V
node function parameter over time, wherein a A-V node function parameter deviating from a reference A-V node function parameter over time indicates incipient A-V node malfunction.

8. The method for detecting incipient A-V node malfunction of a patient according to claim 7, further comprising:
analysing said morphology to determine A-V node function
indicating characteristics, wherein said A-V node function indicating characteristics are a width of a QRS-complex;
determining at least one A-V node function parameter indicating
the function of the A-V node for consecutive cardiac cycles, wherein said at least one A-V node function parameter includes said width of said QRS-complex; and
detecting incipient A-V node malfunction by monitoring said A-V node function parameter over time, wherein an increasing A-V node function over time indicates incipient A-V node malfunction or wherein an A-V node function exceeding a reference A-V node parameter.

9. The method for detecting incipient A-V node malfunction of a patient according to claim 1, further comprising:
detecting incipient A-V node malfunction using said A-V node function parameter, wherein an A-V node function parameter including temporarily poor A-V conduction conditions indicates incipient A-V node malfunction.

10. The method for detecting incipient A-V node malfunction of a patient according to claim 1, further comprising:
sensing at least one physiological or hemodynamical parameter of the patient; and
if said physiological or hemodynamical parameter reaches a predetermined level, temporarily increase the atrial rate a predetermined amount.

11. The method for detecting incipient A-V node malfunction of a patient according to claim 10, further comprising:
sensing the activity level of the patient;
if said activity level exceeds a predetermined level, temporarily increase an atrial rate a predetermined amount; and
detecting incipient A-V node malfunction according to any one of preceding claims at said increased atrial rate.

12. The method for detecting incipient A-V node malfunction of a patient according to claim 1, further comprising:
if an incipient malfunction of the A-V node is detected, notifying said patient by issuing a perceptible alert signal.

13. The method for detecting incipient A-V node malfunction of a patient according to claim 1, further comprising:
if an incipient malfunction of the A-V node is detected, sending a notifying message to an external device.

14. An implantable medical device capable of detecting incipient A-V node malfunction of a patient, said device being connectable to at least one sensor for sensing far-field ventricular events and intrinsic or paced atrial events, said device further comprising:
a cardiac event detection module adapted to determine whether a far-field ventricular event following intrinsic or paced atrial events are sensed during a predetermined period of time;
detect an AV conduction capacity based on whether far-field ventricular events following intrinsic or paced atrial events are sensed, wherein a sensed far-field ventricular event following an intrinsic or paced atrial event during a predetermined period of time indicates good AV conduction capacity and wherein absence of a far-field ventricular event within the predetermined period of time indicates poor AV conduction capacity; and
an A-V node function detection module adapted to determine at least one A-V node function parameter indicating a function of the A-V node, said A-V node function parameter including whether a status of said AV conduction capacity is good or poor; and
detect incipient A-V node malfunction based on said A-V node function parameter.

15. The implantable medical device for detecting incipient A-V node malfunction of a patient according to claim 14, wherein said A-V node function detection module is adapted to:
determine at least one A-V node function parameter indicating a function of the A-V node, said A-V node function parameter including a present paced or sensed atrial rate; and
detect incipient A-V node malfunction based on said A-V node function parameter.

16. The implantable medical device for detecting incipient A-V node malfunction of a patient according to claim 14, wherein said A-V node function detection module is adapted to:
determine at least one A-V node function parameter indicating the function of the A-V node, wherein said A-V node function parameter includes an atrial rate above which the AV conduction capacity becomes poor; and
detect incipient A-V node malfunction by comparing said A-V node function parameter with a reference atrial rate above which the AV conduction capacity becomes poor, wherein an A-V node function parameter being below said reference indicates an incipient A-V node malfunction.

17. The implantable medical device for detecting incipient A-V node malfunction of a patient according to claim 16, wherein said A-V node function detection module is adapted to:
determine at least one A-V node function parameter indicating the function of the A-V node at predetermined intervals, wherein said A-V node function parameter includes an atrial rate above which the AV conduction capacity becomes poor; and
detect incipient A-V node malfunction by monitoring said A-V node function parameter over time, wherein a lowering A-V node function parameter over time indicates an incipient A-V node malfunction.

18. The implantable medical device for detecting incipient A-V node malfunction of a patient according to claim 14, wherein
said cardiac event detection module is adapted to:
determine a PR or AR interval based on sensed ventricular and intrinsic or paced atrial events; and wherein
said A-V node function detection module is adapted to:
determine said at least one A-V node function parameter indicating the function of the A-V node, wherein said at least one A-V node function parameter includes said PR or AR interval as a function of atrial rate; and
detect incipient A-V node malfunction by comparing said A-V node function parameter with a reference PR or AR interval at a present atrial rate, wherein an A-V node function parameter exceeding said reference indicates incipient A-V node malfunction.

19. The implantable medical device for detecting incipient A-V node malfunction of a patient according to claim 18, wherein
said cardiac event detection module is adapted to:
determine PR or AR intervals based on sensed ventricular and intrinsic or paced atrial events at predetermined intervals; and wherein said A-V node function detection module is adapted to:
determine said at least one A-V node function parameter indicating the function of the A-V node for consecutive cardiac cycles, wherein said at least one A-V node function parameter includes said PR or AR interval as a function of atrial rate; and
detect incipient A-V node malfunction by monitoring said A-V node function parameter over time, at specific atrial rates, wherein an increasing A-V node function parameter over time indicates incipient A-V node malfunction.

20. The implantable medical device for detecting incipient A-V node malfunction of a patient according to claim 14, wherein said A-V node function detection module is adapted to:
determine a morphology of said sensed ventricular events;
analyse said morphology to determine A-V node function indicating characteristics;
determine at least one A-V node function parameter indicating the function of the A-V node for consecutive cardiac cycles, wherein said at least one A-V node function parameter includes said A-V node function indicating characteristics; and
detect incipient A-V node malfunction by monitoring said A-V node function parameter over time, wherein a A-V node function parameter deviating from a reference A-V node function parameter over time indicates incipient A-V node malfunction.

21. The implantable medical device for detecting incipient A-V node malfunction of a patient according to claim 20, wherein said A-V node function detection module is adapted to:
analyse said morphology to determine A-V node function indicating characteristics, wherein said A-V node function indicating characteristics are a width of a QRS-complex;
determine at least one A-V node function parameter indicating the function of the A-V node for consecutive cardiac cycles, wherein said at least one A-V node function parameter includes said width of said QRS-complex; and
detect incipient A-V node malfunction by monitoring said A-V node function parameter over time, wherein an increasing A-V node function over time indicates incipient A-V node malfunction or wherein an A-V node function exceeding a reference A-V node parameter.

22. The implantable medical device for detecting incipient A-V node malfunction of a patient according to claim 14, wherein said A-V node function detection module is adapted to:
detect incipient A-V node malfunction using said A-V node function parameter, wherein an A-V node function parameter including temporarily poor A-V conduction conditions indicates incipient A-V node malfunction.

23. The implantable medical device for detecting incipient A-V node malfunction of a patient according to claim 14, further comprising:
a sensor adapted to sense at least one physiological or hemodynamical parameter of the patient; and wherein
a control module is adapted to, if said physiological or hemodynamical parameter reaches a predetermined level, instruct a pulse generator to temporarily increase the atrial rate a predetermined amount; and wherein said A-V node function detection module is adapted to detect incipient A-V node malfunction according to said increased atrial rate.

24. The implantable medical device for detecting incipient A-V node malfunction of a patient according to claim 23, wherein said sensor is an activity sensor adapted to sense the activity level of the patient; and wherein said control module is adapted to, if said activity level exceeds a predetermined level, instruct the pulse generator to temporarily increase an atrial rate a predetermined amount.

25. The implantable medical device for detecting incipient A-V node malfunction of a patient according to claim 14, wherein said A-V node function module is adapted to, if an incipient malfunction of the A-V node is detected, issue an alert signal, which may notify said patient via alert means adapted to issue a perceptible alert signal.

26. The implantable medical device for detecting incipient A-V node malfunction of a patient according to claim 14, wherein said A-V node function module is adapted to, if an incipient malfunction of the A-V node is detected, send a notifying message to an external device via a telemetry module.
